SEQUENCE LISTING (6 sequences)

SEQ ID NO: 1 - protein, 561 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGR

SEQ ID NO: 2 - protein, 484 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGR

SEQ ID NO: 3 - protein, 370 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPP

SEQ ID NO: 4 - protein, 259 aa, Homo sapiens
YLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPV

SEQ ID NO: 5 - protein, 173 aa, Homo sapiens
CMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPV

SEQ ID NO: 6 - protein, 192 aa, Homo sapiens
VQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSF

(19) United States
(12) Patent Application Publication
(10) Pub. No.: US 2019/0383816 A1
(43) Pub. Date: Dec. 19, 2019

(54) METHOD AND COMPOSITION FOR DETECTION OF PROTEOLYTIC PRODUCTS AND DIAGNOSIS OF MALIGNANT NEOPLASTIC DISEASE

(71) Applicants: Vasily Nikolaevich YAKOVLEV, Moscow (RU); Alexei Alexeevich KANAEV, Moscow (RU); Rustam Raisovich SULEIMANOV, Moscow (RU)

(72) Inventors: Vasily Nikolaevich YAKOVLEV, Moscow (RU); Alexei Alexeevich KANAEV, Moscow (RU); Rustam Raisovich SULEIMANOV, Moscow (RU)

(21) Appl. No.: 16/558,259

(22) Filed: Sep. 2, 2019

Related U.S. Application Data
(63) Continuation-in-part of application No. 15/304,896, filed on Oct. 18, 2016, now Pat. No. 10,444,236.

(30) Foreign Application Priority Data
Aug. 25, 2015 (RU) RU20150135793

(51) Int. Cl. G01N 33/574

(52) U.S. Cl. CPC G01N 33/574; G01N 2800/52

(57) ABSTRACT

Tumor invasion and metastasis is accompanied by significant activations of specific proteolysis enzymes. Tumor area is known to have increased infiltration of immunoglobulins G (“IgG”), so IgG may undergo proteolysis in this area. Serine proteases usually cleave peptide bonds between positively charged amino acids lysine and arginine. Since the intact PLG molecules as well as its fragments have lysine binding sites, they can bind to damaged IgG or fragments thereof with free C-terminal lysine that can appear in a circulation after proteolysis in the malignant tumor area. In the present invention we demonstrated the increased binding of damaged IgG or fragments thereof with free C-terminal lysine to fragments of PLG in samples from patients with breast cancer, ovarian cancer, lung cancer, colorectal cancer, prostate cancer vs. samples from healthy donors, and thus we proposed a novel diagnostic method.

### CROSS-REFERENCE TO RELATED APPLICATIONS

The present patent application is a Continuation-in-Part of U.S. patent application Ser. No. 15/304,896, filed Oct. 18, 2016, currently allowed, which is a National stage application from PCT Application PCT/RU2016/000573, filed Aug. 25, 2016, which claims priority to Russian Patent Application RU20150135793, filed Aug. 25, 2015, all of which are incorporated herein in their entireties.

### FIELD OF THE INVENTION

The present invention relates to the detection of malignant neoplastic disease. The invention relates particularly to immunological diagnostic methods that utilize the full-length molecule of plasminogen, or fragments thereof, which may be used as universal detectors of proteolytic products of immunoglobulins G (“IgG”) having a free C-terminal lysine. Furthermore, it relates to a composition and a method for an improved detection of malignant neoplastic disease, as well as diagnostic and/or prognostic uses.

### BACKGROUND OF THE INVENTION

Tumor invasion and metastasis is accompanied by significant activations of specific proteolysis enzymes. This process leads to proteolytic degradation of the extracellular matrix. The serine proteases of the plasminogen activation proteolytic cascade are well known to play an important role in the process of tumor cell invasion.

Serine proteases usually cleave peptide bonds between the positively charged amino acids lysine and arginine, as well as the esters and amides of these amino acids. To date, some authors have shown that the products of proteolytic activity can serve as a universal marker, the detection of which is associated with oncogenic processes. There exists data of the specific proteolysis of immunoglobulins by plasmin (See Peter S. Harpel et al., The Journal of Biological Chemistry Vol. 264, No. 1, Issue of January 5, pp. 616-624 (1989)). Following cleavage, the IgG fragments were shown to specifically interact with plasminogen due to the presence of a C-terminal lysine. IgG fragments that bind to plasminogen were treated with carboxypeptidase B, which specifically cleaves only C-terminals lysine and arginine. After this treatment, the proteins lost their ability to bind to plasminogen, indicating that C-terminal lysine participation is essential for the binding to plasminogen and its fragments. Plasminogen has 5 domains, referred to as “kringles” (K1, K2, K3, K4, and K5), which have a strong affinity for lysine. Each kringle has a strong conservative aminoacid sequence—Asn-Tyr-Cys-Arg-Asn-Pro-Asp—which is a special attribute of kringles of plasminogen.

Since the intact plasminogen molecules as well as its fragments have lysine binding sites, they can bind to proteins with a C-terminal lysine and be used for detection of damaged IgG or IgG fragments in a circulation that appears after proteolisis in the malignant tumor area. These detectors have universal properties compared with other proposed methods of detecting degradation products which require using specific monoclonal antibodies for each product of proteolysis.

Accordingly, it is an object of the present invention to provide means and methods to perform accurate and less biased diagnostic tests in a simple and efficient way for routine testing when diagnosing or prognosing malignant neoplastic diseases.

### SUMMARY OF THE INVENTION

Increased levels of the damaged IgG and/or fragments with a free C-terminal lysine thereof can serve as diagnostic markers of diseases associated with elevated levels of damaged IgG and/or fragments with a free C-terminal lysine thereof. The present invention describes a new method for detection of the damaged IgG and/or fragments with a free C-terminal lysine thereof in a biological fluid. The invention also comprises a diagnostic test system (e.g., kit) for identifying subjects with a high concentration of the damaged IgG and/or fragments with a free C-terminal lysine thereof. This diagnostic test system is comprised of:

The ligand—a fragment of human plasminogen comprised of one sequence of SEQ ID NO: 1-4 (Table 1), at least, including the negative control sample (NC) and positive control sample (PC). The damaged IgG and/or fragments with a free C-terminal lysine thereof bind to fragments of human plasminogen comprised of one sequence of SEQ ID NO: 1-4, and the damaged IgG and/or fragments with a free C-terminal lysine thereof may be detected using, e.g., an ummunoassay technique. One aspect of the present invention is a method for detecting damaged IgG and/or fragments with a free C-terminal lysine thereof in a biological sample, in vitro, the method comprising the steps of:
a) contacting said biological sample with a composition comprising at least one of the four sequences listed in Table 1 (SEQ1-SEQ4) wherein damaged IgG or fragments with a free C-terminal lysine thereof bind to the at least one of four above sequences, and
b) detecting complexes with damaged IgG or fragments with a free C-terminal lysine thereof.

Accordingly, a differential presence of the damaged IgG or fragments with a free C-terminal lysine thereof found in a given biological sample provides useful information regarding the probability of whether a subject being tested has malignant neoplastic diseases, such as but not limited to lung cancer, breast cancer, colorectal cancer, and ovarian cancer. The probability that a subject being tested has a malignant neoplastic disease depends on whether the quantity of the damaged IgG or fragments with a free C-terminal lysine thereof, in a test sample taken from said subject, are statistically significant from a quantity of the damaged IgG or fragments with a free C-terminal lysine thereof in a biological sample taken from healthy subjects, or alternatively statististically significant from a control level known to exist in healthy subjects.

### DETAILED DESCRIPTION OF PREFERRED EMBODIMENTS

### Definitions

Technical and scientific terms used in the description have the same meaning and value that are commonly used in the relevant areas of science and technology, except as further indicated herein below.

The term “antigen”, as used herein, refers to a protein or fragments of a protein, capable of binding antibodies.

The term “ligand”, as used herein, refers to a peptide sequence capable of binding to a free C-terminal lysine.

The term “kringle”, as used herein, refers to a protein domain having a structure stabilized by three disulfide bonds and having a strong conservative aminoacid sequence—Asn-Tyr-Cys-Arg-Asn-Pro-Asp, or its equivalents.

The term “domain”, as used herein, refers to a part of a protein characterized by certain structural or functional properties.

The term “analysis”, as used herein, refers to methods of identifying the molecular compounds, comprising the steps of: (a) the interaction with the antigen within a biological sample under suitable conditions to form an antigen-antibody complex; and (b) the detection of these complexes.

The term “marker”, as used herein, refers to particular molecular compounds of a specific structure, the presence of which, in human tissue samples, is associated with a specific range of diseases.

The term “epitope”, as used herein, refers to a region of a protein molecule which is capable of interacting with the antibody.

The term “detector”, as used herein, refers to a protein molecule which is capable of forming a non-covalent bond with another protein molecule.

The term “diagnostic test”, as used herein, refers to the detection of a diagnostic determinant using a specific laboratory method, the analytical parameters of which remain constant.

The term “subject”, as used herein, includes humans, non-human primates, such as but not limited to chimpanzees and other ape and monkey species, farm animals such as but not limited to cattle, sheep, pigs, goats, and horses, domestic mammals such as but not limited to dogs and cats, laboratory animals including but not limited to rodents such as but not limited to mice, rats, and guinea pigs. The term does not denote a particular age or sex. Thus, adult and newborn subjects, as well as fetuses, whether male or female, are intended to be covered. In preferred embodiments, the subject is a mammal, including humans and non-human mammals. In the most preferred embodiment, the subject is a human.

The term “healthy”, as used herein, refers to a subject possessing good health. Such a subject demonstrates an absence of any malignant or non-malignant disease. In the context of this application, a “healthy individual” is only healthy in that they have an absence of any malignant or non-malignant disease. A “healthy individual” may have other diseases or conditions that would normally not be considered “healthy”.

The term “biological sample”, as used herein, encompasses a variety of sample types obtained from any subject having or not having malignant neoplasm. A typical subject is a human; however, any mammal that has a malignant neoplasm that may develop cancer can serve as a source of a biological sample useful in the disclosed methods. Exemplary biological samples useful in the disclosed methods include but are not limited to clinical samples, cells in culture, cell supernatants, cell lysates, and body fluids. For example, biological samples include samples obtained from fluids collected from an individual suspected of having a malignant neoplasm. Examples of biological fluid samples include but are not limited to blood serum, blood plasma, lymph, exudates, feces, gastric acid, gastric juice, lymph, mucus, pericardial fluid, peritoneal fluid, pleural fluid, pus, saliva, sputum, synovial fluid, tears, sweat, vaginal secretion, vomit, and urine.

The terms “polypeptide,” “protein,” and “peptide”, as used herein interchangeably, refer to amino acid chains in which the amino acid residues are linked by peptide bonds or modified peptide bonds. The amino acid chains can be of any length which is greater than two amino acids. Unless otherwise specified, the terms “polypeptide”, “protein”, and “peptide”, also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching, and cross-linking. Furthermore, amino acids other than the conventional twenty amino acids encoded by genes may also be included in a polypeptide.

The term “lung cancer”, as used herein, refers to a neoplasm, e.g., a malignant neoplasm, of the lung within a given subject, wherein the neoplasm is of epithelial origin (i.e., carcinoma of the lung). Lung carcinomas are categorized by the size and appearance of the malignant cells and the term “lung cancer” includes both non-small cell lung cancer (NSCLC) and small cell lung cancer (SCLC). The term “lung cancer” includes both localized and metastasized lung cancer. The term “lung cancer” can be qualified by the terms “localized” or “metastasized” to differentiate between different types of tumors, where “localized” refers to the original mother tumor, and“metastasized”refers to the tumors that have spread from the original mother tumor.

The TNM System (tumor, node, metastases), as referred to herein, may be used to stage NSCLC in an initial evaluation. Using the TNM descriptors, a group is assigned, ranging from occult cancer, through stages 0, IA (one-A), IB, MA, MB, IMA, NIB and IV (four). This stage group assists with the choice of treatment and the estimation of a prognosis.

For both NSCLC and SCLC, the two general types of staging evaluations are clinical staging and surgical staging. Clinical staging is performed prior to definitive surgery. It is based on the results of imaging studies (such as CT scans and PET scans) and biopsy results. Surgical staging is evaluated either during or after the operation, and is based on the combined results of surgical and clinical findings, including surgical sampling of thoracic lymph nodes

The term “ovarian cancer”, as used herein, refers to a neoplasm, e.g., a malignant neoplasm, of the ovary within a given female subject, wherein the neoplasm is of epithelial origin. The term “ovarian cancer”includes both localized and metastasized ovarian cancer. The term “ovarian cancer” can be qualified by the terms “localized” or “metastasized” to differentiate between different types of tumor, where “localized” refers to the original mother tumor, and “metastasized” refers to tumors that have spread from the original mother tumor.

Ovarian cancer can be staged according to the AJCC/TNM System. This describes the extent of the primary tumor (T), the absence or presence of metastasis to nearby lymph nodes (N), and the absence or presence of distant metastasis (M). The extent of primary tumor contains three sub-categories, T1, T2, T3. This closely resembles the system that is actually used by most gynecologic oncologists, called the FIGO system. Both rely on the results of surgery for the actual stage. In the FIGO system the tumor stage is classified from Stage I-Stage IV (T1-T4) depending on how far the tumor has spread, where stage IV (T4) is worst, meaning that the tumor has spread to its estimated limit. Stages T1-T3 further contain the subcategories, A, B and C.

The term “breast cancer”, as used herein, refers to a neoplasm, e.g., a malignant neoplasm, of the mammary gland within a given female subject, wherein the neoplasm is of epithelial origin. The term “breast cancer”includes both localized and metastasized breast cancer. The term “breast cancer” can be qualified by the terms “localized” or “metastasized” to differentiate between different types of tumor, where “localized” refers to the original mother tumor, and “metastasized” refers to tumors that have spread from the original mother tumor.

Breast cancer can be staged according to the AJCC/TNM System. This describes the extent of the primary tumor (T), the absence or presence of metastasis to nearby lymph nodes (N), and the absence or presence of distant metastasis (M). The extent of primary tumor contains four stages (I-IV), T1, T2, T3, and T4. Diagnosis relies on the results of surgery for the actual stage. Stage IV is worst, meaning that the tumor has spread to its estimated limit. Stages T1-T2, further contain subcategories, A, B, and T3 contain subcategories, A, B, C.

The term “colorectal cancer”, as used herein, refers to a neoplasm, e.g., a malignant neoplasm, of the colon or rectum within a given subject, wherein the neoplasm is of epithelial origin. The term “colorectal cancer”includes both localized and metastasized colorectal cancer. The term “colorectal cancer” can be qualified by the terms “localized” or “metastasized” to differentiate between different types of tumors, where “localized” refers to the original mother tumor, and “metastasized” refers to tumors that have spread from the original mother tumor.

Colorectal cancer can be staged according to the AJCC/TNM System. This describes the extent of the primary tumor (T), the absence or presence of metastasis to nearby lymph nodes (N), and the absence or presence of distant metastasis (M). The extent of primary tumor contains four stages, each with three stages (I-III) T1, T2, T3. Diagnosis relies on the results of surgery for the actual stage. Stage IV is worst, meaning that the tumor has spread to its estimated limit. Stages T1-T2, further contain subcategories, A, B and T3 contain subcategories, A, B, C.

The term “prostate cancer”, as used herein, refers to a neoplasm, e.g., a malignant neoplasm, of the prostate of a given subject, wherein the neoplasm is of epithelial origin. The term “prostate cancer”includes both localized and metastasized prostate cancer. The term “prostate cancer” can be qualified by the terms “localized” or “metastasized” to differentiate between different types of tumors, where “localized” refers to the original mother tumor, and “metastasized” refers to tumors that have spread from the original mother tumor.

Prostate cancer can be staged according to the AJCC/TNM System. This describes the extent of the primary tumor (T), the absence or presence of metastasis to nearby lymph nodes (N), and the absence or presence of distant metastasis (M). The extent of primary tumor contains four main categories, T1, T2, T3, and T4. Diagnosis relies on the results of surgery for the actual stage. Stages T1-T2 further contain subcategories, A, B, C. Stage T3 further contains the subcategories, A and B.

The preferred embodiments of the present invention are described in the following paragraphs.

The method for the preparation of the heavy chain, (Glu-H) Glu1-Arg561, and light chain (Glu-L), Val562-Asn791, of human plasminogen, is described as follows.

The basic method comprises the activation of plasminogen to plasmin, followed by the reduction of S—S bonds between heavy and light chains in conditions that exclude autolysis, then isolating the fragments using affinity chromatography on Lys-Sepharose 4 B. Urokinase cleaves the Arg561-Val562 bond in plasminogen. The resulting plasmin cuts the 77-78 bond and cleaves off the N-terminal peptide (1-77). Mercaptoethanol reduces the two bonds between Cys558-Cys566 and Cys548-Cys666, which link the heavy and light chains.

First step: Glu-plasminogen is isolated from frozen human donor plasma by affinity chromatography on Lys-Sepharose 4 B, at 4° C., and a pH of 8.0. Blood plasma is thawed in the presence of aprotinin, centrifuged for 30 min, at 4° C., and diluted 2-fold in a 0.02 M phosphate buffer, pH 8.0, containing 20 KIU/ml aprotinin. The prepared plasma is then applied onto a Lys-Sepharose 4 B column, equilibrated with a 0.1 M K-phosphate buffer, pH 8.0, containing 20 KIU/ml aprotinin. The column is washed to remove unbound proteins with a 0.3 M phosphate buffer, pH 8.0, containing 20 KIU/ml aprotinin, overnight, to an absorbance at A280=0.05-0.01. Glu-plasminogen is eluted with a solution of 0.2 M 6-aminocaproic acid in a 0.1 M K-phosphate buffer, pH 8.0, containing 20 KIU/ml aprotinin. Fractions containing protein are pooled and subjected to further purification by precipitation (NH₄)₂SO₄ (0.31 g/ml protein solution). The precipitate is stored at 4° C. for 18-24 hours and then separated by centrifugation, and dissolved in a 0.05 M Tris-HCl buffer, pH 8.0, to a concentration of 1.5-2.0 mg/ml. The purified Glu-plasminogen is then dialyzed at 4° C. against water (pH 8.0), and lyophilized.

Second step: Urokinase is added to a final concentration of 600 IU/ml to a solution of Glu-plasminogen (5 mg/ml) in a 0.05 M Tris-HCl buffer, pH 8.8, containing 0.02 M L-lysine, 0.15 M NaCl, 20% glycerol, and 6,000 KIU/ml aprotinin, and incubated for 4 h at 37° C. The progression of conversion of Glu-plasminogen to plasmin is monitored by the hydrolysis of the specific substrate, S-2251 (HD-Val-Leu-Lys p-nitroanilide, Sigma, USA), by plasmin in samples from the reaction, with complete conversion identified by observation of the maximum conversion rate for the substrate.

Third step: This step described the reduction of S—S-bonds between the heavy and light chains of plasmin. Mercaptoethanolis added to the plasmin solution to a final concentration of 0.25 mM and incubated under nitrogen in the dark for 20 minutes at room temperature. The resulting free SH-groups are blocked by adding a freshly prepared solution of iodoacetic acid in a 0.1 M Na-phosphate buffer, pH 8.0 (to a final concentration of 0.315 M), and incubated for 20 minutes.

Fourth step: This step described the separation of the heavy and light chains of plasmin by column chromatography on Lys-Sepharose 4 B. The reaction mixture is diluted to a concentration of 1 mg/ml of protein with 0.1 M Na-phosphate buffer, pH 8.0, containing 20 KIU/ml aprotinin and applied to a Lys-Sepharose 4 B column, equilibrated with the same buffer. Chromatography is performed at 25° C. The heavy chain of plasmin, containing kringles K1-K4 and 30 amino acid residues of the connecting peptide, is adsorbed onto the sorbent. The light chain is washed away with the equilibration buffer. The heavy chain (MR˜56-57 kDa) is eluted with a 0.2 M solution of 6-aminocaproic acid in a 0.1 M Na-phosphate buffer, pH 8.0. The pooled fractions are dialyzed against water (pH˜8.0) and lyophilized.

The purity and molecular weight of the protein were assessed by 12% SDS-polyacrylamide gel electrophoresis. The absence of amidase activity (for S-2251) before and after incubation with urokinase confirmed that the solution of the heavy chain did not contain trace concentrations of miniplasminogen, which may go undetected by electrophoresis.

The purification of Lys-plasminogen (Lys78-Asn791) and its heavy chain (Lys-H Lys78-Arg561) may be performed by the same method, but without aprotinin.

a) The isolation of kringle domains K1-K4 (Tyr80-Ala440), K1-K3 (Tyr80-Val338), and K4-K5 (Val355-Phe546) was performed using elastase treatment of Glu-plasminogen by the method described in the work of Cao and colleagues (See Cao Y., Ji R. W., Davidson D., Schaller J., Marti D., Sohndel S., McCanse S. G., O'Reilly M. S., Llinas M., and Folkman J., J. Biol. Chem., 271, 29461-29467 (1996)). Glu-plasminogen was incubated with elastase at a ratio of 50:1 in a buffer containing 0.05 M Tris-HCl, pH 8.5, 0.5 M NaCl, and 200 KIU aprotinin, for 5 hours at room temperature. The reaction was stopped by adding PMFS to a concentration of 1 mM for 40-50 min. Gel-filtration on a Sephadex G-75 column was performed to separate low and high molecular weight proteins. Protein fractions of the second peak containing K1-3K, K1-K4, K4-K5, and miniplasminogen were applied to Lys-Sepharose 4 B affinity column equilibrated with a buffer containing 0.05 M Tris-HCl, pH 8.5 and 0.15 M NaCl. After the removal of miniplasminogen which was not adsorbed onto the Lys-Sepharose 4 B in the flow-through fraction, the adsorbed fragments K1-K3, K1-K4 and K4-K5 were eluted with a solution of 0.2 M 6-aminocaproic acid in the same buffer, dialyzed against a buffer containing 0.02 M Tris-HCl, pH 8.0, and applied to a column of heparin-agarose equilibrated with the same buffer. Unbound fragments K1-K4 and K4-K5 were eluted with 0.02 M Tris-HCl, pH 8.0,and fragment K1-K3 was eluted with a solution of 0.25 M KCl in the same buffer. The purified fragment K1-K3 was dialyzed against water and lyophilized. Fragments K1-K4 and K4-K5 were separated by gel filtration on Sephadex G-75.Kringle K1 (Tur80-Glu164) and K2-K3 (Cys165-Val338) were isolated from the K1-K3 (Tyr80-Val338) via treatment with pepsin (or protease s.aureus V8) with a further separation on Lys-Sepharose 4 B and gel filtration on Sephadex G-75.

The production of a diagnostic test system (e.g., kit) for ELISA to assay damaged IgG or fragments with (i.e. having)a free C-terminal lysine thereof is described in the following paragraphs. The kit may be used to analyze the test subject's biological samples to perform any of the following non-exclusionary purposes:

i) detecting damaged IgG and/or fragments thereof with a free C-terminal lysine in a biological sample of lung cancer, breast cancer, colorectal cancer, ovarian cancer, and prostate cancer subjects;

ii) detecting lung cancer, breast cancer, colorectal cancer, ovarian cancer, and prostate cancer in a subject; or

iii) diagnosing or prognosing lung cancer, breast cancer, colorectal cancer, ovarian cancer, and prostate cancer in the subject; or

iv) predicting outcome of treatment of lung cancer, breast cancer, colorectal cancer, ovarian cancer, and prostate cancer in the subject; or

v) assessing efficacy of treatment lung cancer, breast cancer, colorectal cancer, ovarian cancer, and prostate cancer in the subject; or

vi) assessing recurrence lung cancer, breast cancer, colorectal cancer, ovarian cancer, and prostate cancer in the subject.

Enzyme-linked immunosorbent assay (ELISA), and sandwich ELISA, are immunoassays that are advantageously used in the methods disclosed herein. In an ELISA, an unknown amount of antigen is affixed to a surface, and then a specific antibody is applied over the surface so that it can bind to the antigen. This antibody is linked to an enzyme, and in the final step, a substance is added so that the enzyme can convert to some detectable signal, most commonly a colour change in a chemical substrate. In a sandwich ELISA, a capture antibody that can bind to the antigen is affixed to the surface. The other steps are equivalent to the ELISA. In an Enzyme Immuno Assay (EIA), which is similar to the sandwich ELISA, streptavidin is affixed to a surface and then the capture antibody is biotinylated, otherwise the other steps are performed equivalently as the ELISA.

When preparing a diagnostic system, fragments of plasminogen containing at least two kringle domains are used as the ligands for coating the solid phase. The various ligands used in ELISA are listed in Table 1. Their primary amino acid sequences are in the sequence listing.

TABLE 1

| peptide chain | Mass, kDa | Name | |
|---|---|---|---|
| Glu¹-Arg⁵⁶¹ | 65 | heavychain (Glu-H) | SEQ ID NO: 1 |
| Lys⁷⁸-Arg⁵⁶¹ | 59 | heavychain (Lys-H) | SEQ ID NO: 2 |
| Lys⁷⁸-Pro⁴⁴⁷ | 58 | K1-4 (Lys⁷⁸-Pro⁴⁴⁷) | SEQ ID NO: 3 |
| Tyr⁸⁰-Val³³⁸ | 41 | Kl-3 (Tyr⁸⁰-Val³³⁸) | SEQ ID NO: 4 |
| Cys¹⁶⁵-Val³³⁸ | 31 | K2-3 (Cys¹⁶⁵-Val³³⁸) | SEQ ID NO: 5 |
| Val³⁵⁵-Phe⁵⁴⁶ | 22 | K4-5 (Val355-Phe546) | SEQ ID NO: 6 |

The ligand was diluted in a 0.1M carbonate-bicarbonate buffer, pH 9.6, at a maximum concentration of 5 microgram/ml.

PBS (phosphate buffered saline): 0.14M NaCl; 0.003 MKCl; 0.005M Na₂HPO₄; 0.002M KH₂PO₄.

Washing solution: 0.05% Tween® 20 in PBS.

Substrate buffer (pH 4.3): 31 mM citric acid, 0.05 N NaOH, 3 mM H₂O₂.

TMB solution: 5 mM 3,3′, 5,5′-tetramethylbenzidine in 70% DMSO.

Chromogenic substrate solution: 4 parts of substrate buffer mixed with 1 part TMB solution.

To create the immunoassay kit, the immobilization of the ligand is preliminarily performed on a solid phase. Various types of carriers for immobilization of a ligand can be used, including cellulose acetate, glass beads, or other particles that can adsorb proteins, as well as immunological plates or plastic strips.

100 microliters of the ligand solution is added to each well of a 96-well immunological plate (Costar). The solution is incubated for 14-16 hours at 4° C. in a humidified chamber. The contents of the wells are discarded. A blocking solution comprising 200 microliters of a 1% solution of bovine serum albumin (BSA) in PBS is added to the wells and incubated for 1.5 to 2 hours at room temperature. After incubation, the blocking solution is removed, the plate is dried overnight at room temperature and then may be used in further applications.

The test sample, negative control samples, and positive control samples are diluted 300-fold with PBS with a 0.5% BSA buffer. 100 microliters of the solution are added to the appropriate wells and incubated for 1 hour at 37° C. After incubation, the solution is discarded, the plate is washed 4 times with the washing solution. 100 microliters of a working solution of the mice monoclonal antibodies to human IgG, conjugated with horseradish peroxidase (Angiogen LLC, Russian Federation), diluted in PBS with 0.5% BSA,is added to the appropriate wells and incubated for 1 hour at 37° C. Unbound components are discarded and the wells are washed with washing solution. 100 microliters of the chromogenic substrate-solution are then added to all the wells and incubated for 15 minutes at 37° C. The reaction is stopped by the addition of 100 microliters of a stop solution (e.g., 2M H₂SO₄). Photometry was performed on a “UNIPLAN” photometer (Pikon, Russia) at a wavelength of 450 nm.

The negative control of concentration level of damaged IgG or fragments with a free C-terminal lysine thereof in ELISA may be performed as follows.

Five sera samples from healthy subjects are taken as negative controls; these were chosen so that the optical density (OD) of each one differed from the group mean by no more than 5% in ELISA. These 5 samples were pooled, and the resulting sample may be used as the negative control sample (NC), taken to indicate the normal (healthy) concentration level of damaged IgG or fragments thereof with a free C-terminal lysine. Dilution of mice monoclonal antibodies to human IgG conjugated with horseradish peroxidase was that OD of C ranged between 0.2 and 0.4. The samples with an OD exceeding that of the NC samples by more than 30% were considered positive. This cutoff range avoids false positives. Positive control (PC) was preapared from human IgG (Sigma-Aldrich) after plasmin treatment. Plasmin-treated IgG was prepared using human IgG by mixing 100 microliters of Lys-plasminogen (1 mg/ml) in PBS with 100 microliters of urokinase (500 IU/ml; Sigma-Aldrich) for 5 minutes, and 100 microliters of human IgG (1 mg/ml in PBS) were added, mixed, and incubated for 6 hours at 37° C. The reaction was stopped by adding of 50 microliters of aprotinin (10,000 IU/ml).

To demonstrate the involvement of C-terminal lysines in binding to plasminogen, the sera samples were diluted by PBS 100-fold and 100 microliters mixed with 2 microliters carboxypeptidase B (CPB; 5 mg/ml; Sigma-Aldrich) in PBS, then incubating for 6 hours at 37° C. The enzyme reaction was stopped by adding 2 microliters of 1,10-phenathroline (Sigma-Aldrich) in methanol (180 mg/ml). The sample of sera was diluted 3-fold and used in ELISA.

The following examples are provided as further clarification of the scope of the present invention. The following examples and any equivalents are disclosed.

Blood samples were drawn from the patients' median cubital veins. The samples of serum were collected. Serum was dispensed out into 100 microliters aliquotes and stored at −40° C.

### EXAMPLE 1

Diagnoses of patients with lung cancer were established on the basis of the following parameters: clinical examination and confirmation by biopsy. The group consisted of 8 patients with cancer, comprising 4 patients with SCLC, stages T2b-T3b, and 4 patients with NSCLC stages T2a-T3a.

ELISA of samples from lung cancer patients was performed according to the method and procedure described herein. Samples wherein the optical density (OD) exceeded the negative control by more than 30% were considered positive.

### Results

Using the following sequences as the ligand: SEQ ID NO1, SEQ ID NO2, SEQ ID NO3, SEQ ID NO4, all 8 samples from lung cancer patients were positive. After incubation of 8 samples from lung cancer patients, at a final dilution of 1:100 with carboxypeptidase B, all 8 samples from lung cancer patients, as well as the positive control, became negative. Using the following sequences as the ligand: SEQ ID NO5 and SEQ ID NO 6 in an ELISA, all 8 samples from lung cancer patients were not differing from positive and negative controls. The OD of these samples did not exceed OD of the negative control plus 30%.

### Conclusion

There is strong correlation between existing lung cancer and a positive value in ELISA according to the present invention. The treatment of the samples by carboxypeptidase B confirms that only C-terminal lysine of damaged IgG or fragments thereof takes part in the binding of damaged IgG or/and fragments with a free C-terminal lysine thereof to: SEQ ID NO1, SEQ ID NO2, SEQ ID NO3, SEQ ID NO4.

ELISA using SEQ ID NO5 and SEQ ID NO 6 did not reveal any difference between the negative control and samples from lung cancer patients, whereas SEQ ID NO5 and SEQ ID NO 6 have only 2 kringles, meaning that at least 3 kringles having a conservative aminoacid sequence—Asn-Tyr-Cys-Arg-Asn-Pro-Asp—are required for detecting damaged IgG or fragments with a free C-terminal lysine thereof.

### EXAMPLE 2

Diagnoses of patients with breast cancer were established on the basis of the following parameters: clinical examination and confirmation by biopsy. The group consisted of 7 patients with breast cancer, stages T2a-T3a.

ELISA of samples from breast cancer patients was performed according to the methods and procedures described herein. Samples where the optical density (OD) exceeded the negative control by more than 30% were considered positive.

### Results

Using the following sequences as the ligand: SEQ ID NO1, SEQ ID NO2, SEQ ID NO3, SEQ ID NO4, all 7 samples from breast cancer patients were positive. After incubation of the 8 samples from breast cancer patients, at a final dilution of 1:100 with carboxypeptidase B, all 8 samples from breast cancer patients, as well as the positive control, became negative.

Using the following sequences as the ligand: SEQ ID NO5 and SEQ ID NO 6 in an ELISA, all 7 samples from breast cancer patients were not differing from positive and negative controls. The OD of these samples did not exceed the OD of the negative control plus 30%.

### Conclusion

There is strong correlation between existing breast cancer and positive value in ELISA of the present invention. The treatment of the samples by carboxypeptidase B means that only C-terminal lysine of damaged IgG or fragments thereof plays a part in the binding of damaged IgG or/and fragments with a free C-terminal lysine thereof to SEQ ID NO1, SEQ ID NO2, SEQ ID NO3, SEQ ID NO4. ELISA using SEQ ID 5 and SEQ ID NO 6 did not reveal any difference between negative control and the samples from breast cancer patients. SEQ ID NO5 and SEQ ID NO 6 have only 2 kringles, which indicates that at least 3 kringles having a conservative aminoacid sequence—Asn-Tyr-Cys-Arg-Asn-Pro-Asp—are needed for detecting damaged IgG or fragments with a free C-terminal lysine thereof.

### EXAMPLE 3

Diagnoses of patients with colorectal cancer were established on the basis of the following parameters: clinical examination and confirmation by biopsy. The group consisted of 8 patients with colorectal cancer, stages T2a-T3b.

ELISA of samples from colorectal cancer patients was performed according to the methods and procedures described herein. Samples where the optical density (OD) exceeded the negative control by more than 30% were considered positive.

### Results

Using the following sequences as the ligand: SEQ ID NO1, SEQ ID NO2, SEQ ID NO3, SEQ ID NO4, all 8 samples from colorectal cancer patients were positive. After incubation of the 8 samples from colorectal cancer patients, at a final dilution of 1:100 with carboxypeptidase B, all 8 samples from colorectal cancer patients as well as the positive control became negative.

Using the following sequences as the ligand: SEQ ID 5 and SEQ ID NO 6 in an ELISA, all 8 samples from colorectal cancer patients were not differing from positive and negative controls. The OD of these samples did not exceed the OD of the negative control plus 30%.

### Conclusion

There is a strong correlation between existing colorectal cancer and positive value in ELISA according to the present invention. The treatment of the samples by carboxypeptidase B means that only C-terminal lysine of damaged IgG or fragments thereof plays a part in the binding of damaged IgG or/and fragments with a free C-terminal lysine thereof to SEQ ID NO1, SEQ ID NO2, SEQ ID NO3, and SEQ ID NO4. ELISA using SEQ ID 5 and SEQ ID NO 6 did not reveal any difference between the negative control and the samples from colorectal cancer patients. SEQ ID NO5 and SEQ ID NO 6 have only 2 kringles, indicating that at least 3 kringles having a conservative aminoacid sequence—Asn-Tyr-Cys-Arg-Asn-Pro-Asp—are required for detecting damaged IgG or fragments with a free C-terminal lysine thereof.

### EXAMPLE 4

Diagnoses of patients with ovarian cancer were established on the basis of the following parameters: clinical examination and confirmation by biopsy. The group consisted of 6 patients with ovarian cancer, stages T2c-T3a.

ELISA of samples from ovarian cancer patients was performed according to the method and procedure described the present invention. The samples where the optical density (OD) exceeded the negative control by more than 30% were considered positive.

### Results

Using the following sequences as the ligand: SEQ ID NO1, SEQ ID NO2, SEQ ID NO3, SEQ ID N04, all 6 samples from ovarian cancer patients were positive. After incubation of the 6 samples from ovarian cancer patients, at a final dilution of 1:100 with carboxypeptidase B, all 6 samples from ovarian cancer patients, as well as the positive control, became negative. Using the following sequences as the ligand: SEQ ID 5 and SEQ ID NO 6 in an ELISA, all 6 samples from ovarian cancer patients were not differing from positive and negative controls. The OD of these samples did not exceed the OD of the negative control plus 30%.

### Conclusion

There is strong correlation between existing ovarian cancer and positive value in ELISA of the present invention. The treatment of the samples by carboxypeptidase B means that only C-terminal lysine of damaged IgG or fragments thereof plays a part in the binding of damaged IgG or/and fragments with a free C-terminal lysine thereof to SEQ ID NO1, SEQ ID NO2, SEQ ID NO3, and SEQ ID NO4. ELISA using SEQ ID 5 and SEQ ID NO 6 did not reveal any difference between the negative control and the samples from ovarian cancer patients. SEQ ID NO5 and SEQ ID NO 6 have only 2 kringles, indicating that at least 3 kringles having a conservative aminoacid sequence—Asn-Tyr-Cys-Arg-Asn-Pro-Asp—are needed for detecting damaged IgG or fragments with a free C-terminal lysine thereof.

### EXAMPLE 5

Diagnoses of patients with prostate cancer were established on the basis of the following parameters: clinical examination, transrectal ultrasonography, and confirmation by biopsy. The group consisted of 10 patients with prostate cancer, stages T2a-T3 a.

ELISA of samples from prostate cancer patients was performed according to the method and procedure described the present invention. The samples where the optical density (OD) exceeded the negative control by more than 30% were considered positive.

### Results

Using the following sequences as the ligand: SEQ ID NO1, SEQ ID NO2, SEQ ID NO3, SEQ ID NO4, all 10 samples from prostate cancer patients were positive. After incubation of the 10 samples from prostate cancer patients, at a final dilution of 1:100 with carboxypeptidase B, all 10 samples from prostate cancer patients, as well as the positive control, became negative.

Using the following sequences as the ligand: SEQ ID 5 and SEQ ID NO 6 in an ELISA, all 10 samples from prostate cancer patients were not differing from positive and negative controls.

### Conclusion

There is strong correlation between existing prostate cancer and a positive value in ELISA according to the present invention. The treatment of the samples by carboxypeptidase B means that only C-terminal lysine of damaged IgG or fragments thereof plays a part the binding of damaged IgG and/or fragments with a free C-terminal lysine thereof to SEQ ID NO1, SEQ ID NO2, SEQ ID NO3, and SEQ ID NO4. ELISA using SEQ ID 5 and SEQ ID NO 6 did not reveal any difference between the negative control and the samples from prostate cancer patients. SEQ ID NO5 and SEQ ID NO 6 have only 2 kringles, indicating that at least 3 kringles having a conservative aminoacid sequence—Asn-Tyr-Cys-Arg-Asn-Pro-Asp—are needed for detecting damaged IgG or fragments with a free C-terminal lysine thereof.

The description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

### SEQUENCE LISTING

<210>1
<212> protein
<213> Homo sapiens
<400>Description of the sequenceSEQ ID NO: 1
Glu¹ Pro Leu Asp Asp Tyr Val AsnThr Gln¹⁰Gly Ala Ser Leu Phe Ser Val Thr Lys Lys²⁰Gln
Leu Gly Ala Gly Ser Ile Glu Glu Cys³⁰ Ala Ala Lys Cys Glu Glu Asp Glu Glu Phe⁴⁰ThrCysArg
Ala PheGln Tyr His Ser Lys⁵⁰ Glu GlnGlnCys Val Ile Met Ala Glu Asn⁶⁰Arg Lys Ser Ser Ile
IleIleArg Met Arg⁷⁰ Asp Val Val Leu Phe Glu Lys Lys⁷⁸ Val Tyr⁸⁰ Leu Ser Glu Cys Lys
ThrGlyAsnGly Lys⁹⁰Asn Tyr ArgGlyThr Met Ser Lys Thr Lys¹⁰⁰AsnGly Ile ThrCysGln Lys
Trp Ser Ser¹¹⁰Thr Ser Pro His Arg Pro ArgPhe Ser Pro¹²⁰ Ala Thr His Pro Ser Glu Gly Leu Glu
Glu¹³⁰Asn Tyr CysArgAsn Pro Asp Asn Asp Pro¹⁴⁰GlnGly Pro TrpCys Tyr ThrThr Asp Pro¹⁵⁰
Glu Lys Arg Tyr Asp Tyr Cys Asp Ile Leu¹⁶⁰ Glu Cys Glu GluGluCys Met His Cys Ser¹⁷⁰Gly
Glu Asn Tyr Asp Gly Lys Ile Ser Lys¹⁸⁰Thr Met Ser Gly Leu Glu CysGln Ala Trp⁸⁰ Asp Ser
Gln Ser Pro His Ala His Gly Tyr²⁰⁰ Ile Pro Ser Lys Phe Pro Asn Lys Asn Leu²¹⁰ Lys LysAsn
Tyr CysArgAsn Pro Asp Arg²²⁰ Glu Leu Arg Pro TrpCysPheThrThr Asp²³⁰ Pro Asn Lys
ArgTrp Glu Leu Cys Asp Ile²⁴⁰ Pro ArgCysThrThr Pro ProPro Ser Ser²⁵⁰Gly Pro Thr Tyr
GlnCys Leu Lys Gly Thr²⁶⁰Gly Glu Asn Tyr ArgGlyAsn Val Ala Val Thr Val Ser Gly His
ThrCysGln His Trp²⁸⁰ Ser Ala GlnThr Pro His Thr His Asn Arg²⁹⁰Thr Pro Glu AsnPhe Pro Cys
Lys Asn Leu³⁰⁰ Asp Glu Asn Tyr CysArgAsn Pro Asp Gly³¹⁰ Lys Arg Ala Pro TrpCys His
ThrThr Asn³²⁰ Ser Gln Val ArgTrp Glu Tyr Cys Lys Ile³³⁰ Pro Ser Cys Asp Ser Ser Pro Val
Ser Thr³⁴⁰ Glu Gln Leu Ala Pro Thr Ala Pro Pro Glu³⁵⁰ Leu Thr Pro Val ValGln Asp Cys Tyr
His³⁶⁰Gly Asp GlyGln Ser Tyr ArgGlyThr Ser³⁷⁰ Ser ThrThrThrThrGly Lys LysCys Gln³⁸⁰ Ser
Trp Ser Ser Met Thr Pro His Arg His³⁹⁰Gln Lys Thr Pro Glu Asn Tyr Pro Asn Ala⁴⁰⁰Gly Leu
Thr Met Asn Tyr CysArgAsn Pro⁴¹⁰ Asp Ala Asp Lys Gly Pro TrpCysPhe Thr⁴²⁰Thr Asp Pro
Ser Val ArgTrp Glu Tyr Cys⁴³⁰Asn Leu Lys LysCys Ser GlyThr Glu Ala⁴⁴⁰ Ser Val Val Ala
Pro ProPro Val Val Leu⁴⁵⁰ Leu Pro Asp Val Glu Thr Pro Ser Glu Glu⁴⁶⁰ Asp Cys Met
PheGlyAsnGly Lys Gly Tyr⁴⁷⁰ArgGly Lys Arg Ala ThrThr Val Thr Gly⁴⁸⁰Thr Pro CysGln Asp
Trp Ala AlaGln Glu⁴⁹⁰Pro His Arg His Ser Ile PheThr Pro Glu⁵⁰⁰ThrAsn Pro Arg Ala Gly Leu
Glu Lys Asn⁵¹⁰ Tyr CysArgAsn Pro Asp Gly Asp Val Gly⁵²⁰Gly Pro TrpCys Tyr ThrThrAsn
Pro Arg⁵³⁰Lys Leu Tyr Asp Tyr Cys Asp Val Pro Gln⁵⁴⁰Cys Ala Ala Pro Ser Phe Asp CysGly
Lys⁵⁵⁰Pro Gln Val Glu Pro Lys LysCys Pro Gly⁵⁶⁰Arg
<210>2
<212> protein
<213> Homo sapiens
<400>Description of the sequenceSEQ ID NO: 2
Lys⁷⁸ Val Tyr⁸⁰ Leu Ser Glu Cys Lys ThrGlyAsnGly Lys⁹⁰Asn Tyr ArgGlyThr Met Ser Lys
Thr Lys¹⁰⁰AsnGly Ile ThrCysGln Lys Trp Ser Ser¹¹⁰Thr Ser Pro His Arg Pro ArgPhe Ser Pro¹²⁰
Ala Thr His Pro Ser Glu Gly Leu Glu Glu¹³⁰Asn Tyr CysArgAsn Pro Asp Asn Asp
Pro¹⁴⁰GlnGly Pro TrpCys Tyr ThrThr Asp Pro¹⁵⁰ Glu Lys Arg Tyr Asp Tyr Cys Asp Ile Leu¹⁶⁰
Glu Cys Glu GluGluCys Met His Cys Ser¹⁷⁰Gly Glu Asn Tyr Asp Gly Lys Ile Ser Lys¹⁸⁰Thr
Met Ser Gly Leu Glu CysGln Ala Trp⁸⁰ Asp Ser Gln Ser Pro His Ala His Gly Tyr²⁰⁰ Ile Pro
Ser Lys Phe Pro Asn Lys Asn Leu²¹⁰ Lys LysAsn Tyr CysArgAsn Pro Asp Arg²²⁰ Glu Leu Arg
Pro TrpCysPheThrThr Asp²³⁰ Pro Asn Lys ArgTrp Glu Leu Cys Asp Ile²⁴⁰ Pro ArgCysThrThr
Pro ProPro Ser Ser²⁵⁰Gly Pro Thr Tyr GlnCys Leu Lys Gly Thr²⁶⁰Gly Glu Asn Tyr ArgGlyAsn
Val Ala Val Thr Val Ser Gly His ThrCysGln His Trp²⁸⁰ Ser Ala GlnThr Pro His Thr His Asn
Arg²⁹⁰Thr Pro Glu AsnPhe Pro Cys Lys Asn Leu³⁰⁰ Asp Glu Asn Tyr CysArgAsn Pro Asp
Gly³¹⁰Lys Arg Ala Pro TrpCys His ThrThr Asn³²⁰ Ser Gln Val ArgTrp Glu Tyr Cys Lys Ile³³⁰
Pro Ser Cys Asp Ser Ser Pro Val Ser Thr³⁴⁰ Glu Gln Leu Ala Pro Thr Ala Pro Pro Glu³⁵⁰ Leu
Thr Pro Val ValGln Asp Cys Tyr His³⁶⁰Gly Asp GlyGln Ser Tyr ArgGlyThr Ser³⁷⁰ Ser
ThrThrThrThrGly Lys LysCys Gln³⁸⁰ Ser Trp Ser Ser Met Thr Pro His Arg His³⁹⁰Gln Lys Thr
Pro Glu Asn Tyr Pro Asn Ala⁴⁰⁰Gly Leu Thr Met Asn Tyr CysArgAsn Pro⁴¹⁰ Asp Ala Asp Lys
Gly Pro TrpCysPhe Thr⁴²⁰Thr Asp Pro Ser Val ArgTrp Glu Tyr Cys⁴³⁰Asn Leu Lys LysCys
Ser GlyThr Glu Ala⁴⁴⁰ Ser Val Val Ala Pro ProPro Val Val Leu⁴⁵⁰ Leu Pro Asp Val Glu Thr
Pro Ser Glu Glu⁴⁶⁰ Asp Cys Met PheGlyAsnGly Lys Gly Tyr⁴⁷⁰ArgGly Lys Arg Ala ThrThr
Val Thr Gly⁴⁸⁰Thr Pro CysGln Asp Trp Ala AlaGln Glu⁴⁹⁰ Pro His Arg His Ser Ile PheThr Pro
Glu⁵⁰⁰ThrAsn Pro Arg Ala Gly Leu Glu Lys Asn⁵¹⁰ Tyr CysArgAsn Pro Asp Gly Asp Val
Gly⁵²⁰Gly Pro TrpCys Tyr ThrThrAsn Pro Arg⁵³⁰ Lys Leu Tyr Asp Tyr Cys Asp Val Pro
Gln⁵⁴⁰Cys Ala Ala Pro Ser Phe Asp CysGly Lys⁵⁵⁰ Pro Gln Val Glu Pro Lys LysCys Pro
Gly⁵⁶⁰Arg
<210>3
<212> protein
<213> Homo sapiens
<400>Description of the sequenceSEQ ID NO: 3
Lys⁷⁸ Val Tyr⁸⁰ Leu Ser Glu Cys Lys ThrGlyAsnGly Lys⁹⁰Asn Tyr ArgGlyThr Met Ser Lys
Thr Lys¹⁰⁰AsnGly Ile ThrCysGln Lys Trp Ser Ser¹¹⁰Thr Ser Pro His Arg Pro ArgPhe Ser Pro¹²⁰
Ala Thr His Pro Ser Glu Gly Leu Glu Glu¹³⁰Asn Tyr CysArgAsn Pro Asp Asn Asp
Pro¹⁴⁰GlnGly Pro TrpCys Tyr ThrThr Asp Pro¹⁵⁰ Glu Lys Arg Tyr Asp Tyr Cys Asp Ile Leu¹⁶⁰
Glu Cys Glu GluGluCys Met His Cys Ser¹⁷⁰Gly Glu Asn Tyr Asp Gly Lys Ile Ser Lys¹⁸⁰Thr
Met Ser Gly Leu Glu CysGln Ala Trp⁸⁰ Asp Ser Gln Ser Pro His Ala His Gly Tyr²⁰⁰ Ile Pro
Ser Lys Phe Pro Asn Lys Asn Leu²¹⁰ Lys LysAsn Tyr CysArgAsn Pro Asp Arg²²⁰ Glu Leu Arg
Pro TrpCysPheThrThr Asp²³⁰ Pro Asn Lys ArgTrp Glu Leu Cys Asp Ile²⁴⁰ Pro ArgCysThrThr
Pro ProPro Ser Ser²⁵⁰Gly Pro Thr Tyr GlnCys Leu Lys Gly Thr²⁶⁰Gly Glu Asn Tyr ArgGlyAsn
Val Ala Val Thr Val Ser Gly His ThrCysGln His Trp²⁸⁰ Ser Ala GlnThr Pro His Thr His Asn
Arg²⁹⁰Thr Pro Glu AsnPhe Pro Cys Lys Asn Leu³⁰⁰ Asp Glu Asn Tyr CysArgAsn Pro Asp
Gly³¹⁰ Lys Arg Ala Pro TrpCys His ThrThr Asn³²⁰ Ser Gln Val ArgTrp Glu Tyr Cys Lys Ile³³⁰
Pro Ser Cys Asp Ser Ser Pro Val Ser Thr³⁴⁰ Glu Gln Leu Ala Pro Thr Ala Pro Pro Glu³⁵⁰ Leu
Thr Pro Val ValGln Asp Cys Tyr His³⁶⁰Gly Asp GlyGln Ser Tyr ArgGlyThr Ser³⁷⁰ Ser
ThrThrThrThrGly Lys LysCys Gln³⁸⁰ Ser Trp Ser Ser Met Thr Pro His Arg His³⁹⁰Gln Lys Thr
Pro Glu Asn Tyr Pro Asn Ala⁴⁰⁰Gly Leu Thr Met Asn Tyr CysArgAsn Pro⁴¹⁰ Asp Ala Asp Lys
Gly Pro TrpCysPhe Thr⁴²⁰Thr Asp Pro Ser Val ArgTrp Glu Tyr Cys⁴³⁰Asn Leu Lys LysCys
Ser GlyThr Glu Ala⁴⁴⁰ Ser Val Val Ala Pro ProPro
<210>4
<212> protein
<213> Homo sapiens
<400>Description of the sequenceSEQ ID NO: 4
Tyr⁸⁰ Leu Ser Glu Cys Lys ThrGlyAsnGly Lys⁹⁰Asn Tyr ArgGlyThr Met Ser Lys Thr
Lys¹⁰⁰AsnGly Ile ThrCysGln Lys Trp Ser Ser¹¹⁰Thr Ser Pro His Arg Pro ArgPhe Ser Pro¹²⁰
Ala Thr His Pro Ser Glu Gly Leu Glu Glu¹³⁰Asn Tyr CysArgAsn Pro Asp Asn Asp
Pro¹⁴⁰GlnGly Pro TrpCys Tyr ThrThr Asp Pro¹⁵⁰ Glu Lys Arg Tyr Asp Tyr Cys Asp Ile Leu¹⁶⁰
Glu Cys Glu GluGluCys Met His Cys Ser¹⁷⁰Gly Glu Asn Tyr Asp Gly Lys Ile Ser Lys¹⁸⁰Thr
Met Ser Gly Leu Glu CysGln Ala Trp⁸⁰ Asp Ser Gln Ser Pro His Ala His Gly Tyr²⁰⁰ Ile Pro
Ser Lys Phe Pro Asn Lys Asn Leu²¹⁰ Lys LysAsn Tyr CysArgAsn Pro Asp Arg²²⁰ Glu Leu Arg
Pro TrpCysPheThrThr Asp²³⁰ Pro Asn Lys ArgTrp Glu Leu Cys Asp Ile²⁴⁰ Pro ArgCysThrThr
Pro ProPro Ser Ser²⁵⁰Gly Pro Thr Tyr GlnCys Leu Lys Gly Thr²⁶⁰Gly Glu Asn Tyr ArgGlyAsn
Val Ala Val Thr Val Ser Gly His ThrCysGln His Trp²⁸⁰ Ser Ala GlnThr Pro His Thr His Asn
Arg²⁹⁰Thr Pro Glu AsnPhe Pro Cys Lys Asn Leu³⁰⁰ Asp Glu Asn Tyr CysArgAsn Pro Asp
Gly³¹⁰ Lys Arg Ala Pro TrpCys His ThrThr Asn³²⁰ Ser Gln Val ArgTrp Glu TyrCys Lys Ile³³⁰
Pro Ser Cys Asp Ser Ser Pro Val
<210>5
<212> protein
<213> Homo sapiens
<400>Description of the sequenceSEQ ID NO: 5
Cys Met His Cys Ser¹⁷⁰Gly Glu Asn Tyr Asp Gly Lys Ile Ser Lys¹⁸⁰Thr Met Ser Gly Leu Glu
CysGln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys LysAsn Tyr CysArgAsn Pro Asp Arg²²⁰ Glu Leu Arg Pro
TrpCysPheThrThr Asp²³⁰ Pro Asn Lys ArgTrp Glu Leu Cys Asp Ile²⁴⁰ Pro ArgCysThrThr Pro
ProPro Ser Ser²⁵⁰Gly Pro Thr Tyr GlnCys Leu Lys Gly Thr²⁶⁰Gly Glu Asn Tyr ArgGlyAsn Val
Ala Val Thr Val Ser Gly His ThrCysGln His Trp²⁸⁰ Ser Ala GlnThr Pro His Thr His Asn
Arg²⁹⁰Thr Pro Glu AsnPhe Pro Cys Lys Asn Leu³⁰⁰ Asp Glu Asn Tyr CysArgAsn Pro Asp
Gly³¹⁰ Lys Arg Ala Pro TrpCys His ThrThr Asn³²⁰ Ser Gln Val ArgTrp Glu TyrCys Lys Ile³³⁰
Pro Ser Cys Asp Ser Ser Pro Val
<210>6
<212> protein
<213> Homo sapiens
<400>Description of the sequence SEQ ID NO: 6
Val Gln Asp Cys Tyr His³⁶⁰Gly Asp GlyGln Ser Tyr ArgGlyThr Ser³⁷⁰ Ser
ThrThrThrThrGly Lys LysCys Gln³⁸⁰ Ser Trp Ser Ser Met Thr Pro His Arg His³⁹⁰Gln
Lys Thr Pro Glu Asn Tyr Pro Asn Ala⁴⁰⁰Gly Leu Thr Met Asn Tyr CysArgAsn Pro⁴¹⁰
Asp Ala Asp Lys Gly Pro TrpCysPhe Thr⁴²⁰Thr Asp Pro Ser Val ArgTrp Glu Tyr
Cys⁴³⁰Asn Leu Lys LysCys Ser GlyThr Glu Ala⁴⁴⁰ Ser Val Val Ala Pro ProPro Val Val
Leu⁴⁵⁰ Leu Pro Asp Val Glu Thr Pro Ser Glu Glu⁴⁶⁰ Asp Cys Met PheGlyAsnGly Lys Gly
Tyr⁴⁷⁰ArgGly Lys Arg Ala ThrThr Val Thr Gly⁴⁸⁰Thr Pro CysGln Asp Trp Ala AlaGln
Glu⁴⁹⁰ Pro His Arg His Ser Ile PheThr Pro Glu⁵⁰⁰ThrAsn Pro Arg Ala Gly Leu Glu Lys
Asn⁵¹⁰ Tyr CysArgAsn Pro Asp Gly Asp Val Gly⁵²⁰Gly Pro TrpCys Tyr ThrThrAsn Pro
Arg⁵³⁰ Lys Leu Tyr Asp Tyr Cys Asp Val Pro Gln⁵⁴⁰Cys Ala Ala Pro Ser Phe

What claimed is:

1. A method for detecting damaged immunoglobulins G (IgG) and/or fragments thereof having a free C-terminal lysine, in a biological sample, the method comprising:
contacting the biological sample with one or more fragments of plasminogen, wherein said fragment(s) of plasminogen comprise one more sequences comprising SEQ ID NOS1-4, and further comprising at least three copies of aminoacid sequence, Asn-Tyr-Cys-Arg-Asn-Pro-Asp, and
detecting complexes comprising damaged IgG and/or fragments thereof, with a free C-terminal lysine,
said detecting comprising comparing an expression of the contacted biological sample to positive and negative controls,
said the biological sample is selected from a group consisting of lung cancer, breast cancer, colorectal cancer, ovarian cancer, and prostate cancer subjects.

2. The method according to claim 1, wherein the biological sample is a biological fluid sample of blood serum, blood plasma.

3. The method according to claim 1, wherein said one or more fragments of plasminogen comprising at least three copies of the aminoacid sequence, Asn-Tyr-Cys-Arg-Asn-Pro-Asp.

4. The method of claim 1, wherein said contacting comprises contacting the biological fluid sample with a solid support, wherein said fragments of plasminogen SEQ ID NOS. 1-4 are immobilized on a surface of the solid support.

5. The method of claim 1, wherein said detecting further comprises using an enzyme-linked immunosorbent assay (ELISA).

6. The method according to claim 1, wherein the positive control comprises damaged IgG and/or fragments thereof, with a free C-terminal lysine.

7. The method according to claim 1, wherein the negative control comprises an absence of damaged IgG and/or fragments thereof with a free C-terminal lysine.

8. The method according to claim 1, the method being performed on an automated reading device.

9. The method according to claim 1, wherein the contacting is performed manually.

12. The method according to claim 1, wherein an increase in a level of damaged IgG and/or fragments thereof with a free C-terminal lysine exceeds 30% relative to the negative control group is indicative of any of the following:(i) diagnosing and/or prognosing malignant neoplastic disease in a subject, (ii) predicting efficacy of treatment of malignant neoplastic disease in a subject, (iii) assessing outcome of treatment of malignant neoplastic disease in a subject, and/or (iv) assessing recurrence of malignant neoplastic disease in a subject wherein the subject is a mammal having, or suspected of having, a malignant neoplastic disease.

13. The method according to claim 12, wherein the malignant neoplastic disease is lung cancer, breast cancer, colorectal cancer, overian cancer, and prostate cancer.

14. A kit for detecting damaged immunoglobulins G (IgG) and/or fragments thereof having a free C-terminal lysine, in a biological sample, the kit comprising:
a 96-well immunological plate coated by at least one fragment of plasminogen comprising SEQ. ID. NO. 1-4,
a positive control sample, said positive control sample comprising damaged IgG and/or fragments thereof with a free C-terminal lysine,
a negative control sample, said negative control sample comprising an absence of damaged IgG and/or fragments thereof having a C-terminal lysine,
one or more mice monoclonal antibodies to human IgG, said mice monoclonal antibodies being conjugated with a horseradish peroxidase,
said kit being used for detecting damaged IgG and/or fragments thereof with a free C-terminal lysine in a biological sample from a test subject, and for further analyzing the test subject having, or suspected of having, a malignant neoplastic disease.
